(19) United States
(12) Patent Application Publication
(10) Pub. No.: US 2011/0183926 A1
(43) Pub. Date: Jul. 28, 2011

(54) TREATMENT USING CONTINUOUS LOW DOSE APPLICATION OF SUGAR ANALOGS

(75) Inventors: Theodore J. Lampidis, Miami, FL (US); Metin Kurtoglu, Miami, FL (US)

(73) Assignee: UNIVERSITY OF MIAMI, Miami, FL (US)

(21) Appl. No.: 12/994,265

(22) PCT Filed: May 26, 2009
(86) PCT No.: PCT/US2009/045157

§ 371 Date: Nov. 23, 2010

Related U.S. Application Data
(60) Provisional application No. 61/071,907, filed on May 23, 2008.

(51) Int. Cl. A61K 31/7004; A61P 35/00

(52) U.S. Cl. 514/23

(57) ABSTRACT

Methods and uses of low dosage nonmetabolizable D-glucose analogs and mannose analogs such as 2-deoxy-D-glucose, 5-thio-D-glucose, 2-fluoro-2-deoxy-D-glucose, 2-chloro-2-deoxy-D-glucose, 2-bromo-2-deoxy-D-glueose, 2-deoxy-2-fluoro-mannose, 2-deoxy-2-chloro-mannose, 3-deoxy mannose, 4-deoxy mannose, and 2,3 didioxy mannose, for the treatment of tumors.

This application claims priority to U.S. provisional application no. 61/071,907, filed May 23, 2008, which is incorporated herein by reference.

The research leading to this invention was supported by a grant from the United States Department of Health and Human Services, National Cancer Institute, CA037109. the U.S. Government has certain rights in the invention.

### BACKGROUND

Cancer cells growing in the hypoxic regions of tumor cells produce energy by glycolytic utilization of glucose and thereby, inhibition of this metabolic pathway by 2-deoxy-D-glucose (2-DG) elicits cytotoxicity in portions of solid tumors that are devoid of adequate oxygenation (hypoxia). Furthermore, the 6 carbon skeleton of glucose molecule is used by tumor cells for forming the sugar backbones of DNA and RNA precursors as well as oligosachharides that are essential for synthesis of glycoproteins. Thus, enhanced glucose consumption is essential for maintaining the high demand of nucleic acid and glycoprotein synthesis in rapidly proliferating tumor cells which underlies the growth inhibitory effect of 2-DG in cancer cells undergoing rapid cell cycle in the presence of sufficient oxygen supply. Taken together, it suggests that 2-DG can be used as a single chemotherapeutic agent as a result of its combined effects on both normoxic and hypoxic regions of solid tumors.

Although in in vitro studies, it was shown that 2-DG is growth inhibitory and toxic in aerobic and anaerobic tumor cells, respectively, in vivo studies have failed to demonstrate the efficacy of this anti-metabolite agent when given as a single chemotherapeutic drug (Kurtoglu M, Maher J C, Lampidis T J. Differential toxic mechanisms of 2-deoxy-D-glucose versus 2-fluorodeoxy-D-glucose in hypoxic and normoxic tumor cells. Antioxid Redox Signal. 2007 Sep; 9(9):1383-90. Review.) An explanation for failure of 2-DG treatment comes from the inventors' observation in in vitro experiments that 24 h continuous application of this drug at a relatively high dose is required for its significant growth inhibitory effect on aerobic tumor cells. Therefore, previous in vivo applications of 2-DG treatment appear not to have achieved effective concentrations and durations of this agent in plasma. In in vivo experiments and phase I clinical trials, 2-DG was given as a bolus oral ingestion or intraperitoneal injection. Since 2-DG is a glucose mimetic, it follows that single oral or intraperitoneal 2-DG administration will yield high concentration of this agent in portal circulation resulting in its sequestration in liver cells due to their glucokinase activity. Moreover, rapid increase of 2-DG concentration in systemic circulation is shown to induce an insulin response, which will lead to greater intracellular accumulation of this glucose analog in adipocytes and muscle cells. Thus, following bolus administration of 2-DG, tumor cells will receive relatively small amounts of 2-DG for a short period of time which we contend are below that necessary to effectively inhibit tumor growth.

### SUMMARY

Tumors are treated by the administration of low continuous doses of nonmetabolizable D-glucose analogs, for example 2-DG.

To achieve a therapeutic effect, patients are treated, for example, with continuous-slow release of 2-DG by a pump system whereby the drug is administered either intraperitoneally, subcutaneously or intravenously, or a transdermal patch or a slow-releasing pill or any other means by which the drug can be released continuously at a low dosage. The dose should maintain a plasma concentration of 2-DG that is, for example, below the Kₘ of liver glucokinase and below that which will induce an insulin response and thereby eventually achieve a therapeutic dose. In general, such dosages will be between 1 μg/ml/hr and 175 μg/ml/hr, for example, 19 μg/ml/hr administered continuously for periods of 1-10 weeks, e.g. for 4 weeks. In one example, this results in the delivery of about 30 mg/kg over a 24 hour period for a period of 4 weeks. Persons of skill in the art will be able to determine suitable dosages for individual patients without undue experimentation.

Accordingly, the invention includes a method of treating a benign or malignant tumor by the continuous administration of low but effective doses of 2-DG, e.g. by an infusion pump. Suitable infusion pumps include for example, intraperitoneal osmotic pumps.

Also included are the use of such nonmetabolizable D-glucose analogs and mannose derivatives to treat tumors and their use in the manufacture of medicaments for the treatment of tumors as described herein.

Other nonmetabolizable D-glucose analogs should also be useful in the invention. Examples are 5-thio-D-glucose, 2-halogen substituted D-glucose analogs such as 2-fluoro-2-deoxy-D-glucose (2-FG), 2-chloro-2-deoxy-D-glucose (2-CG), 2-bromo-2-deoxy-D-glucose (2-BG). Additional examples of suitable analogs are described in U.S. Pat. No. 6,670,330. Mannose derivatives/analogs, such as 2-deoxy-2-fluoro- mannose (2-FM) and 2-deoxy-2-chloro-mannose (2-CM), 3-deoxy mannose, 4-deoxy mannose, and 2,3 didioxy mannose, as described, for example, in WO2007/100728, are also expected to be useful.

The nonmetabolizable D-glucose analog or mannose derivative may also be administered with other chemotherapeutic agents, particularly in the case of drug resistant tumors. Examples of additional chemotherapeutic agents include adriamycin, vinblastine, paclitaxel, and vincristine

The treatment method described herein can be used for treating any disease that would benefit from inhibition of glucose metabolism including cancer, viral infections, psoriasis and obesity. In such diseases there is an increased glucose metabolism for synthesis of various intracellular metabolites, i.e. glycoproteins in viruses, nucleic acids in psoriasis and lipid vacuoles in obesity. Therefore, it is expected that inhibition of glucose metabolism should impact these pathogenic mechanisms.

### DETAILED DESCRIPTION

The invention provides methods for treating cancerous and noncancerous tumors in an animal, e.g. a mammal, particularly a human.

The term “cancerous tumor” is intended to include any malignant tumor that may or may not have undergone metastasis. The term “noncancerous tumor” is intended to include any benign tumor. These terms are used as customarily understood by persons of skill in the art.

Tumors to be treated include, inter alia, any cancerous or noncancerous tumor that exhibits drug resistance, in particular drug resistance/multidrug resistance caused by the presence/overexpression of an MDR1 gene in the cells (Ling, V. Multidrug resistance: Molecular Mechanisms and Clinical Relevance, Cancer Chemother. Pharmacol. 40:53-58 1997). Such tumors include, inter alia, breast cancer, metastatic carcinoma of the lung, primary melanoma, ovarian cancer, multiple myeloma, and Non-Hodgkin's Lymphoma.

By “multidrug resistant” or “MDR” is meant cells, microorganisms, etc. that arc resistant to one or more therapeutic compounds intended to inactivate or kill those cells or microorganisms, including those that, for example, exhibit high MDR-1 gene expression.

Additional examples of benign and malignant tumors which may be treated by the

compositions and methods of the invention can be found in Table 1-1 of Cancer Biology (Raymond W. Ruddon, Cancer Biology, 3rd Ed., Oxford Univ. Press, 1995, incorporated herein by reference). Tumors to be treated include those that are known to be of viral origin, as well as those that are not of viral origin. The compositions and methods of the invention are expected to be particularly useful in the treatment of solid tumors as well as hematological malignancies and sarcomas.

As used herein, “about” is intended to mean +/−10%.

By “pharmaceutically acceptable diluents, excipients and carriers” is meant such compounds as will be known to persons of skill in the art as being compatible with the pharmaceutical compositions and suitable for local or systemic administration to an animal, particularly a human or other mammal, according to the invention.

As used herein, the terms “treatment,” “treating,” etc., refer to obtaining a desired pharmacologic and/or physiologic effect. The effect may be prophylactic in terms of completely or partially preventing a condition or disease or symptom thereof and/or may be therapeutic in terms of a partial or complete cure for a condition or disease and/or any adverse affect attributable to the condition or disease. “Treatment,” thus, for example, covers: (a) preventing the condition or disease from occurring in an individual who is predisposed to the condition or disease but has not yet been diagnosed as having it; (b) inhibiting the condition or disease, such as, arresting its development; and (c) relieving, alleviating or ameliorating the condition or disease, such as, for example, causing regression of the condition or disease.

The term “pharmaceutically acceptable carrier” refers to a non-toxic solid, semisolid or liquid filler, diluent, encapsulating material or formulation auxiliary of any conventional type. A “pharmaceutically acceptable carrier” is non-toxic to recipients at the dosages and concentrations employed, and is compatible with other ingredients of the formulation. For example, the carrier for a formulation containing the present therapeutic compounds and compositions preferably does not include oxidizing agents and other compounds that are known to be deleterious to such. Suitable carriers include, but are not limited to, water, dextrose, glycerol, saline, ethanol, buffer, dimethyl sulfoxide, Cremaphor EL, and combinations thereof. The carrier may contain additional agents such as wetting or emulsifying agents, or pH buffering agents. Other materials such as anti-oxidants, humectants, viscosity stabilizers, and similar agents may be added as necessary.

Pharmaceutically acceptable salts herein include the acid addition salts (e.g. formed with a free amino group) and which are formed with inorganic acids, including, but not limited to hydrochloric or phosphoric acids, or such organic acids as acetic, mandelic, oxalic, and tartaric. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, and histidine.

The term “pharmaceutically acceptable excipient,” includes vehicles, adjuvants, or diluents or other auxiliary substances, such as those conventional in the art, which are readily available to the public. For example, pharmaceutically acceptable auxiliary substances include pH adjusting and buffering agents, tonicity adjusting agents, stabilizers, wetting agents and the like.

As used herein, the singular forms “a”, “an”, and “the” include plural forms unless the context clearly dictates otherwise. Thus, for example, reference to “a compound” includes a plurality of such compounds.

As mentioned above, effective amounts of the pharmaceutical compounds are administered to an individual, where “effective amount” means a dosage sufficient to produce a desired result. In some embodiments, the desired result is arrest of growth of, or shrinkage of a tumor.

Typically, the compositions to be used in the instant invention will contain from less than about 1% up to about 99% of the active ingredient(s). The appropriate dose to be administered depends on the subject to be treated, such as the general health of the subject, the age of the subject, the state of the disease or condition, the weight of the subject, etc.

The pharmaceutically acceptable excipients, such as vehicles, adjuvants, carriers or diluents, are conventional in the art. Suitable excipient vehicles are, for example, water, saline, dextrose, glycerol, ethanol, or the like, and combinations thereof. In addition, if desired, the vehicle may contain minor amounts of auxiliary substances such as pH adjusting and buffering agents, tonicity adjusting agents, stabilizers, wetting agents or emulsifying agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the individual being treated.

The therapeutic compounds can be formulated into preparations for injection by dissolving, suspending or emulsifying them in an aqueous or non-aqueous solvent, such as vegetable or other similar oils, including corn oil, castor oil, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Patents and other publications cited herein are hereby incorporated by reference.

### Example 1

Recently we have produced in vivo data which supports this idea and teaches that 19 μg/ml/hr or 30 mg/kg per 24 hrs continuously for 4 weeks effectively reduces tumor burden in an orthotopic human breast cancer mouse model.

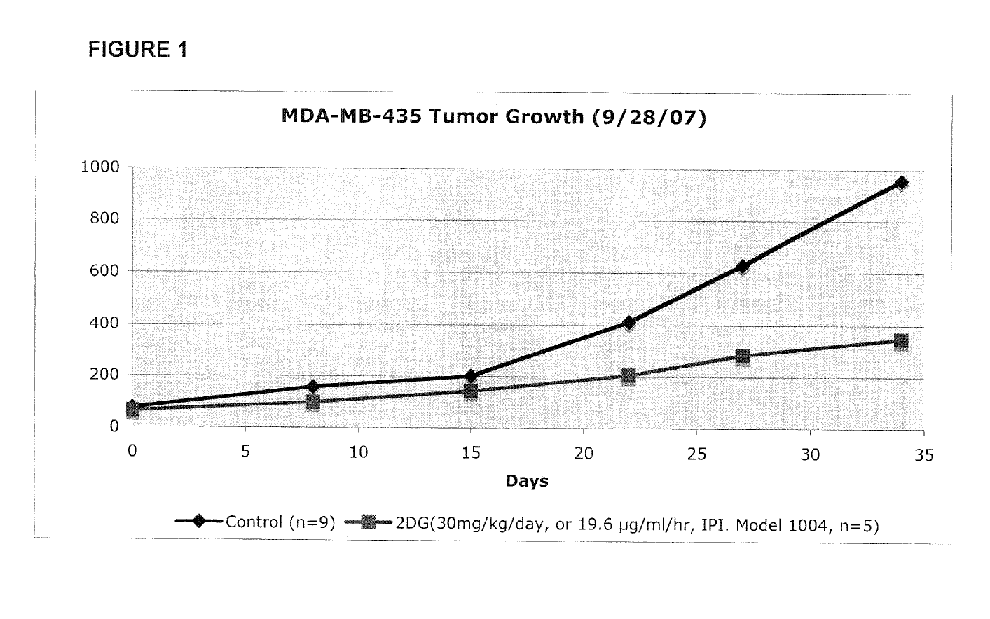

2×10⁶ MDA-MB-435 cells were injected orthotopically to CD-1 nude mice. After two weeks, osmotic pumps (Model 1004, Alzet Inc.) were inserted into the peritoneal cavity under general anesthesia. In one group of animals (n=9), the pumps contained 0.9% NaCl, while in the other group (n=5), the pumps were filled with 2-DG at a concentration of 328 mg/ml. Mice were weighed, and tumor measurements were taken by caliper three times weekly. Tumor measurements were converted to tumor volume by using the formula W×L²/2. Mice were killed when either W or L exceeded 15 mm. At sacrifice, mice were weighed, and tumors were excised and checked histologically for verification of tumor growth. The results are shown in FIG. 1.

By using the present invention, tumor control and/or eradication will be improved. Similarly, any disease which can be treated by inhibiting glucose metabolism should benefit from this invention by avoiding an insulin response and/or through sequestration by the liver and thereby increasing the ability to achieve an effective dose.

### REFERENCES CITED

1. Ahren B and Hedner P. Mechanism for the inhibitory action of 2-deoxy-glucose on thyroid hormone secretion in the mouse. Neuroendocrinology 49: 471/175, 1989.
2. Brown J. Effects of 2-deoxyglucose on carbohydrate metabolism:review of the literature and studies in the rat. Metabolism 11:1098-1112, 1962.
3. Karlsson S and Ahren B. Inhibition of 2-deoxy-glucose-induced glucagon secretion by muscarinic and alpha-adrenoceptor blockade in the mouse. Diabetes Res Clin Pract 3: 239-242, 1987.
4. Karlsson S, Bood M, and Ahren B. The mechanism of 2-deoxyglucose-induced insulin secretion in the mouse. J Auton Pharmacol 7: 135-144, 1987.
5. Landau B R, Laszlo J, Stengle J, and Burk D. Certain metabolic and pharmacologic effects in cancer patients given infusion of 2-deoxy-D-glucose. J Natl Cancer Inst 21: 485-494, 1958.
6. Taborsky G J Jr, Halter J B, and Porte D Jr. Morphine suppressesplasma catecholamine responses to laparotomy but not to 2-deoxyglucose. Am J Physiol 242: E317-E322, 1982.
7. U.S. Pat. No. 6,670,330 (Theodore J Lampidis) Cancer chemotherapy with 2-deoxy-D-glucose
8. U.S. Pat. No. 6,979,675 (George Tidmarsch) Treatment of cancer with 2-deoxyglucose.

1. A method of treating a tumor in a patient in need thereof, said method comprising administering a continuous low therapeutically effective dose of a nonmetabolizable D-glucose analog or mannose analog, said low therapeutically effective dose being below that which will induce an insulin response in said patient.

2. The method of claim 1 wherein the nonmetabolizable D-glucose analog is 2-deoxy-D-glucose (2-DG).

3. The method of claim 1 wherein the nonmetabolizable D-glucose analog or mannose analog is administered by a route selected from the group consisting of intraperitoneally, subcutaneously or intravenously, or a transdermal patch or a slow- releasing pill.

4. The method of claim 1, wherein the effective dosage produces a plasma concentration of the nonmetabolizable D-glucose analog or mannose analog that is below the Kn, of liver glucokinase.

5. The method of claim 1, wherein the dosage is between 1 g/ml/hr and 175 g/ml/hr.

6. The method of claim 1, wherein the dosage is between 4 and 20 g/ml/hr.

7. The method of claim 1, wherein the dosage is about 19 g/ml/hr.

8. The method of claim 1, wherein the dosage is administered continuously for periods of 1-10 weeks.

9. The method of claim 8 wherein the dosage is administered for at least 4 weeks.

10. The method of claim 1, wherein the tumor is a malignant tumor.

11. The method of claim 1, wherein the malignant tumor is a solid tumor.

12. A composition for the treatment of a tumor in a subject comprising a nonmetabolizable D-glucose analog or mannose analog.

13. The composition of claim 12, wherein said composition is formulated in a dosage that is below that which will induce an insulin response in said subject.

14. The composition of claim 12, wherein the nonmetabolizable D-glucose analog is 2-deoxy-D-glucose (2-DG).

15. The composition of claim 12, wherein the formulation is suitable for administration by a route selected from the group consisting of intraperitoneally, subcutaneously or intravenously, or a transdermal patch or a slow-releasing pill.

16. The composition of claim 14, wherein the formulation is suitable for administration by a route selected from the group consisting of intraperitoneally, subcutaneously or intravenously, or a transdermal patch or a slow-releasing pill.

17. A composition for treating a tumor in a subject, comprising a nonmetabolizable D-glucose analog or mannose analog.

18. The composition of claim 17, wherein said nonmetabolizable D-glucose analog or mannose analog is administered in a dosage that is below that which will induce an insulin response in said subject.

19. The composition of claim 17, wherein the nonmetabolizable D-glucose analog is 2-deoxy-D-glucose (2-DG).

20. The composition of claim 17, wherein the nonmetabolizable D-glucose analog or mannose analog is administered by a route selected from the group consisting of intraperitoneally, subcutaneously or intravenously, or a transdermal patch or a slow-releasing pill.

21. The composition of claim 17, wherein the 2-deoxy-D-glucose (2-DG) is administered by a route selected from the group consisting of intraperitoneally, subcutaneously or intravenously, or a transdermal patch or a slow-releasing pill.

22. The method of claim 1 wherein the nonmetabolizable D-glucose analog or mannose analog is selected from the group consisting of 5-thio-D-glucose, 2-fluoro-2- deoxy-D-glucose (2-FG), 2-chloro-2-deoxy-D-glucose (2-CG), 2-bromo-2-deoxy-D-glucose (2-BG), 2-deoxy-2-fluoro-mannose (2-FM), 2-deoxy-2-chloro-mannose (2- CM), 3-deoxy mannose, 4-deoxy mannose, and 2,3 didioxy mannose.

23. The composition of claim 12, wherein the nonmetabolizable D-glucose analog or mannose analog is selected from the group consisting of 5-thio-D-glucose, 2-fluoro-2- deoxy-D-glucose (2-FG), 2-chloro-2-deoxy-D-glucose (2-CG), 2-bromo-2-deoxy-D- glucose (2-BG), 2-deoxy-2-fluoro-mannose (2-FM), 2-deoxy-2-chloro-mannose (2-CM), 3-deoxy mannose, 4-deoxy mannose, and 2,3 didioxy mannose.

24. The composition of claim 17, wherein the nonmetabolizable D-glucose analog or mannose analog is selected from the group consisting of 5-thio-D-glucose, 2-fluoro-2- deoxy-D-glucose (2-FG), 2-chloro-2-deoxy-D-glucose (2-CG), 2-bromo-2-deoxy-D- glucose (2-BG), 2-deoxy-2-fluoro- mannose (2-FM), 2-deoxy-2-chloro-mannose (2-CM), 3-deoxy mannose, 4-deoxy mannose, and 2,3 didioxy mannose.
